(19) United States
(12) Patent Application Publication
(10) Pub. No.: US 2006/0246592 A1
(43) Pub. Date: Nov. 2, 2006

(54) Identification of low vapor pressure toxic chemicals

(75) Inventor: Ram A. Hashmonay, Chapel Hill, NC (US)

Correspondence Address: JENKINS, WILSON, TAYLOR & HUNT, P. A., 3100 TOWER BLVD, SUITE 1200, DURHAM, NC, 27707, US

(73) Assignee: ARCADIS G&M, Inc.

(21) Appl. No.: 11/100,329

(22) Filed: Apr. 6, 2005

Related U.S. Application Data
(60) Provisional application No. 60/559,879, filed on Apr. 6, 2004.

(51) Int. Cl. G01N 33/00

(52) U.S. Cl. 436/57

(57) ABSTRACT

The presently disclosed subject matter relates to methods, systems, and computer program products for monitoring for low vapor pressure noxious compounds in the atmosphere. More particularly, the presently disclosed subject matter relates to an active, modulated open-path infrared method, system, and computer program product for detecting, identifying, and quantifying one or more low vapor pressure noxious compounds in the atmosphere, wherein the one or more low vapor pressure compounds can be present in the vapor phase, the aerosol phase, adsorbed on airborne particulate matter, and combinations thereof.

### CROSS REFERENCE TO RELATED APPLICATIONS

This application is based on and claims priority to U.S. Provisional Patent Application Ser. No. 60/559,879, filed Apr. 6, 2004, which is incorporated herein by reference in its entirety.

### TECHNICAL FIELD

The presently disclosed subject matter relates to methods, systems, and computer program products for monitoring for low vapor pressure noxious compounds in the atmosphere. More particularly, the presently disclosed subject matter relates to an active, modulated open-path infrared method, system, and computer program product for detecting, identifying, and quantifying one or more low vapor pressure noxious compounds in the atmosphere, wherein the one or more low vapor pressure compounds can be present in the vapor phase, the aerosol phase, adsorbed on airborne particulate matter, and combinations thereof.

### ABBREVIATIONS

AEGL=acute exposure guideline level
CLS=classical-least-squares
CWA=chemical warfare agent
DAAMS=depot area air monitoring system
FTIR=Fourier transform infrared
g=gram
IDLH=immediately dangerous to life and health
IR=infrared
K=degrees Kelvin
m=meter
MCT=mercury-cadmium-telluride
mg=milligram
NRT=near real-time
OP=open path
OP-IR=open-path infrared
OP-FTIR=open-path Fourier transform infrared
ORS=optical remote sensing
OSHA=Occupational Safety and Health Administration
PM=particulate matter
ppb=parts per billion
ppm=parts per million
ppm-m=parts per million-meter
QC=quantum cascade
TDL=tunable diode laser
TIC=toxic industrial chemical
pg=microgram
μm=micrometer

### BACKGROUND

Optical remote sensing (ORS) techniques have been used in a variety of environmental monitoring applications, including the measurement of noxious compounds emitted from smoke stacks, landfills, and other fugitive emission sources. See generally, Grant et al., J. Air Waste Manage. Assoc., 42, 18 (1992). More particularly, open-path Fourier transform infrared (OP-FTIR) techniques, in either the active or passive measurement modes, have been used to detect and identify noxious compounds in the gas phase, in which the delivery mechanism of the noxious compound is diffusion into the air. See Russwurm. G. M. and Childers, J. W., Open-path Fourier Transform Infrared Spectroscopy, in Handbook of Vibrational Spectroscopy, Vol. 2 (Chalmers, J. M., and Griffiths, P. R., eds., John Wiley & Sons, Ltd.), pp. 1750-1773 (2002).

The use of ORS systems, including OP-FTIR systems, to monitor for noxious compounds in the gas phase, however, does not provide any warning of potential human exposure in the event that a noxious compound, such as a toxic industrial chemical, an agricultural chemical, e.g., a pesticide, a chemical warfare agent, or a bioaerosol, is released as an aerosol or is adsorbed on airborne particulate matter either prior to or subsequent to being released. Sensors based on optical spectroscopic techniques, such as OP-FTIR systems, are either not capable of or have not been conditioned to respond to low vapor pressure compounds, e.g., compounds with a vapor pressure of about 10⁻² Torr or less, in the aerosol phase or low vapor pressure compounds adsorbed on airborne particulate matter.

Further, the use of ORS techniques to simultaneously detect the presence of low vapor pressure compounds in the vapor phase, aerosol phase, and when adsorbed on airborne particulate matter has not been demonstrated. Thus, there is a need in the art for improved methods for detecting, identifying, and quantifying low vapor pressure noxious compounds in the atmosphere, whether the low vapor pressure noxious compound is in the vapor phase, aerosol phase, adsorbed on airborne particulate matter, or combinations thereof.

### SUMMARY

The presently disclosed subject matter provides an active, modulated open-path infrared method, system, and computer program product for detecting, identifying, and quantifying one or more low vapor pressure noxious compounds in the atmosphere, wherein the one or more low vapor pressure compounds can be present in the vapor phase, the aerosol phase, adsorbed on airborne particulate matter, and combinations thereof.

In some embodiments, the method for monitoring for one or more low vapor pressure compounds in the atmosphere comprises:
(a) providing an instrument adapted for emitting modulated infrared radiation along a monitoring path;
(b) providing at least one detector disposed so as to detect the modulated infrared radiation emitted by the instrument, wherein the detector is capable of producing a signal indicative of an apparent absorption spectrum of the low vapor pressure compound;
(c) positioning the instrument such that the emitted modulated infrared radiation traverses the monitoring path;
(d) measuring the apparent absorption spectrum of the low vapor pressure compound, wherein the apparent absorption spectrum exhibits two or more characteristics selected from the group consisting of:
   (i) one or more absorption bands;
   (ii) one or more a derivative-like features;
   (iii) one or more wavelength dependent baseline offsets; and
   (iv) combinations thereof; and
(e) correlating the two or more characteristics to provide one of:
   (i) a detection;
   (ii) an identification;
   (iii) a quantification; and
   (iv) combinations thereof;
      of one or more low vapor pressure compounds to monitor the one or more low vapor pressure compounds in the atmosphere.

In some embodiments, the presently disclosed subject matter provides a system for monitoring for one or more low vapor pressure compounds in the atmosphere, the system comprising:
(a) an instrument adapted for emitting modulated infrared radiation along a monitoring path;
(b) at least one detector disposed so as to detect the modulated infrared radiation emitted by the instrument, wherein the detector is capable of producing a signal indicative of the apparent absorption spectrum of the low vapor pressure compound, and wherein the apparent absorption spectrum exhibits two or more characteristics selected from the group consisting of:
   (i) one or more absorption bands;
   (ii) one or more derivative-like features;
   (iii) one or more wavelength dependent baseline offsets; and
   (iv) combinations thereof;
(c) a memory in which a plurality of machine instructions are stored; and
(d) at least one processor that is coupled to the at least one detector and the memory, wherein the processor is capable of executing the plurality of machine instructions stored in the memory, causing the processor to:
   (i) record the signal indicative of an apparent absorption spectrum of the low vapor pressure compound, wherein the apparent absorption spectrum exhibits two or more characteristics selected from the group consisting of one or more absorption bands, one or more derivative-like features; one or more wavelength dependent baseline offsets; and combinations thereof; and
   (ii) correlate the two or more characteristics to provide one of a detection; an identification; a quantification; and combinations thereof of one or more low vapor pressure compounds to monitor one or more low vapor pressure compounds in the atmosphere.

In some embodiments, the instrument comprises an active, modulated open-path infrared (OP-IR) spectrometer system. In some embodiments, the OP-IR spectrometer system comprises an open-path Fourier transform infrared (OP-FTIR) system. In some embodiments, the OP-IR spectrometer system is in the monostatic configuration. In some embodiments, the OP-IR spectrometer system comprises a pulsed quantum cascade (QC) laser infrared radiation source. One of ordinary skill in the art would recognize, however, that the presently disclosed methods, systems, and computer program products would be applicable to any active optical remote sensing (ORS) technique known in the art in which the infrared radiation source is modulated.

In some embodiments, the presently disclosed subject matter provides a computer program product comprising computer-executable instructions embodied in a computer-readable medium for performing steps comprising:
(a) inputting a signal indicative of an apparent absorption spectrum of a low vapor pressure compound, wherein the apparent absorption spectrum exhibits two or more characteristics selected from the group consisting of an absorption band, a derivative-like feature, a wavelength dependent baseline offset, and combinations thereof; and
(b) correlating the two or more characteristics to provide one of:
   (i) a detection;
   (ii) an identification;
   (iii) a quantification; and
   (iv) combinations thereof,
to monitor for one or more low vapor pressure compounds in the atmosphere.

Thus, the presently disclosed methods, systems, and computer program products are capable of detecting, identifying, and quantifying low vapor pressure compounds which are present in a vapor phase, an aerosol phase, adsorbed on airborne particulate matter, and combinations thereof. Low vapor pressure compounds for which the presently disclosed method is applicable include, but are not limited to, noxious compounds, such as industrial toxic chemicals, agricultural chemicals, e.g., pesticides, chemical warfare agents, and bioaerosols. In some embodiments, the noxious compound comprises a low vapor pressure organophosphate compound, such as a chemical warfare agent, including, but not limited to O-ethyl-S-(2-iisopropylaminoethyl)methyl phosphonothiolate (VX), ethyl N,N-dimethylphosphoroamidocyanidate (GA), and O-cyclohexyl-methylphosphonofluoridate (GF). Indeed the presently disclosed methods, systems, and computer program products are applicable to any low vapor pressure compound that exhibits one or more absorption bands, one or more derivative-like features, and/or one or more wavelength dependent baseline offsets in the mid-infrared spectral region, e.g., from about 5000 cm⁻¹ to about 500-cm⁻¹.

By correlating the two or more characteristics of the apparent absorption spectrum recorded by the open-path infrared system, low vapor pressure compounds in the vapor phase, aerosol phase, and adsorbed on airborne particulate matter can be distinguished. In doing so, the presently disclosed methods, systems, and computer program products can increase the accuracy of the identification of the one or more noxious compounds and can decrease the likelihood of false positives as compared to approaches currently available in the art. Further, the presently disclosed method also can be used to generate data in real time to provide a warning of potential hazardous exposure to the one or more low vapor pressure noxious compounds.

The presently disclosed methods, systems, and computer program products can be used to monitor for the release of one or more low vapor pressure noxious compounds along a fenceline, e.g., the property line and/or an outer boundary, of a facility having one or more noxious chemicals disposed therein, such as a chemical plant or a chemical weapon stockpile, or to monitor along the fenceline of a permanent or semi-permanent facility that houses one or more human occupants, such as a civilian residential area, a military base, or a military camp.

Accordingly, it is an object of the presently disclosed subject matter to provide a novel method, system, and computer program product for detecting, identifying, and quantifying low vapor pressure noxious compounds in the atmosphere. This and other objects are achieved in whole or in part by the presently disclosed subject matter.

An object of the presently disclosed subject matter having been stated hereinabove, other objects and aspects will become evident as the description proceeds when taken in connection with the accompanying Drawings and Examples as best described herein below.

### BRIEF DESCRIPTION OF THE DRAWINGS

FIGS. 1A-1C are schematic representations of active, modulated open-path infrared (OP-IR) systems suitable for use with the presently disclosed subject matter.

FIG. 1A is an active, modulated monostatic OP-IR system in which the active, modulated infrared source and the detector are positioned at the same end of the monitoring path and the transmitted optical beam and the returned optical beam travel along substantially an identical path.

FIG. 1B is an active, modulated monostatic OP-IR system in which the active, modulated infrared source and the detector are positioned at the same end of the monitoring path, and wherein the transmitted optical beam is translated such that the returned optical beam traverses a path that is offset with respect to the path traversed by the transmitted optical beam.

FIG. 1C is an active, modulated bistatic OP-IR system in which the IR source and the detector are positioned at opposite ends of the monitoring path.

FIGS. 2A and 2B are schematic representations of open-path infrared systems in which the infrared source, either an active infrared source or ambient background radiation, is not modulated before the optical beam is transmitted along the monitoring path.

FIG. 2A is an active bistatic OP-IR system in which the IR source and the detector are positioned at opposite ends of the monitoring path.

FIG. 2B is a passive OP-IR system in which the ambient background in the field of view of the receiving optics supplies the infrared radiation that interrogates the plume.

FIG. 3 shows the estimated detection limits of an OP-IR system for sulfur hexafluoride as a function of radiation source temperature.

FIGS. 4A-4C are representative infrared spectra of malathion.

FIG. 4A is an open-path infrared (OP-IR) spectrum of aerosolized malathion dispersed in the atmosphere. FIG. 4B is a portion of the spectrum shown in FIG. 4A expanded in the fingerprint region of the mid-infrared spectral region (900 cm⁻¹ to 1100 cm⁻¹) to show a derivative-like spectral feature characteristic of malathion. FIG. 4C compares the derivative-like spectral features of the OP-IR spectrum of aerosolized malathion dispersed in the atmosphere (solid line) with the absorption bands of vapor phase malathion (dotted line) in the 790 cm⁻¹ to 1090 cm⁻¹ spectral region.

FIG. 5 shows simulated extinction spectra for VX aerosol in the 950- to 1100-cm¹ (9.1- to 10.5-μm) spectral region for three different size distributions, wherein m=10, δ=5 (dashed line); m=5, δ=5 (dotted line); m=2, δ=5 (thin solid line); and the extinction spectrum of aerosolized malathion (thick solid line), and wherein m is the mean distribution and δ is the standard deviation.

FIG. 6 shows representative OP-IR spectra of airborne dust particles (dotted line), aerosolized malathion (thick solid line), and malathion adsorbed on airborne dust particles (thin solid line).

### DETAILED DESCRIPTION

The presently disclosed subject matter will now be described more fully hereinafter with reference to the accompanying Examples and Drawings, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

I. Definitions

As used herein, the terms “open-path monitoring” and “optical remote sensing” are used interchangeably and refer to monitoring over a location in space, i.e., a “monitoring path” or “a line of measurement,” that is completely open to the atmosphere.

An “optical remote sensing monitor” refers to an optical system comprising an energy source, i.e., a radiation source, such as an infrared source or an ultraviolet source, capable of emitting energy along a path and at least one detector capable of detecting the energy emitted by the energy source, wherein the detector produces a signal indicative of the path-integrated concentration of the species of interest along the path. For an overview of optical remote sensing monitors and methods of use thereof, see ASTM E-1865-97, Standard Guide for Open-Path Fourier Transform Infrared (OP/FT-IR) Monitoring of Gases and Vapors in Air; ASTM E 1982-98, Standard Practice for Open-Path Fourier Transform Infrared (OP/FT-IR) Monitoring of Gases and Vapors in Air; and U.S. Pat. No. 6,542,242 to Yost et al., each of which is incorporated herein by reference in its entirety.

An “active” ORS system refers to an ORS system that comprises an energy source, such as an infrared source or an ultraviolet source, which supplies the optical beam to be transmitted along the monitoring path.

A “passive” ORS system refers to an ORS system that relies on energy emitted from a blackbody radiation source in the field of view of the receiving optics to supply the optical beam which interrogates, for example, a plume comprising one or more noxious compounds. A passive ORS system also can be used to measure the emission spectra of noxious compounds in a plume, when the temperature of the plume is greater than the temperature of the ambient background.

The term “optical beam” refers to the energy emitted by an ORS instrument. In most ORS instruments, the energy emitted by the source, e.g., an infrared source, is collimated by reflecting optics before it is transmitted along the monitoring path.

A “bistatic system” refers to an optical system in which the radiation source, e.g., an infrared source, is positioned some distance from a detector. In ORS systems, this term generally means that the energy source and the detector are at opposite ends of the monitoring path.

A “monostatic system” refers to an optical system in which the radiation source and the detector are positioned at the same end of the monitoring path. In monostatic ORS systems, the optical beam generally is returned to the detector by a reflecting element, such as a retroreflector.

A “retroreflector” refers to an optical device that returns radiation, e.g., an optical beam, in a direction substantially the same as the direction from which it came. Retroreflectors come in a variety of forms. The type of retroreflector typically used in ORS measurements comprises three mutually perpendicular surfaces with which to return the optical beam in a direction substantially the same as the direction from which it came. This type of retroreflector is referred to as a “cube-corner retroreflector.”

The term “monitoring path” refers to the location in space over which the presence of a gas, vapor, aerosol, particle, or combinations thereof, is monitored.

The term “monitoring pathlength” refers to the distance over which the optical beam traverses through the monitoring path.

The term “parts per million meters” refers to the units associated with the quantity “path-integrated concentration” and is a possible unit of choice for reporting data from ORS monitors. The unit is abbreviated as “ppm-m” and is independent of the monitoring pathlength.

The term “path-integrated concentration” refers to the quantity measured by an ORS system along the monitoring path. The path-integrated concentration is expressed in units of concentration times length, for example ppm-m and is independent of the monitoring pathlength.

The term “path-averaged concentration” refers to the result of dividing the path-integrated concentration by the pathlength. The path-averaged concentration gives the average value of the concentration along the path, and typically is expressed in units of parts per million (ppm), parts per billion (ppb), or micrograms per cubic meter (μg/m³).

A “plume” refers to the gaseous and/or aerosol effluents emitted from an emission source, e.g., a pollutant source, such as a smoke stack or a landfill, and the volume of space the gaseous and/or aerosol effluents occupy.

The term “aerosol” refers to a gaseous suspension of fine solid or liquid particles.

The term “vapor” refers to the gaseous state of a substance that is a liquid or a solid under standard temperature and pressure.

The term “noxious compound” refers to a compound that is harmful, injurious, and/or unpleasant to a living thing. Representative noxious compounds include, but are limited to, odorous compounds and toxic compounds, such as toxic industrial chemicals, agricultural chemicals, e.g., pesticides, chemical warfare agents, and bioaerosols.

The phrase “immediately dangerous to life and health” or “IDLH” is defined by the Occupational Safety and Health Administration (OSHA) as an atmospheric concentration of any toxic, corrosive, or asphyxiant substance that poses an immediate threat to life, causes irreversible or delayed adverse health effects, or interferes with an individual's ability to escape from danger. Thus, in some embodiments, noxious compounds are IDLH compounds.

The phrase “acute exposure guideline level” or “AEGL” describes the dangers to humans resulting from short-term exposure to airborne chemicals.

The term “target species” refers to a compound, such as, but not limited to, a noxious compound as defined hereinabove, including odorous compounds and toxic compounds, such as toxic industrial chemicals, agricultural chemicals, e.g., pesticides, chemical warfare agents, and bioaerosols, for which instrumental parameters are selected and analysis methods are developed to detect, identify, and/or quantify the target species in the atmosphere.

The terms “monitor” and “monitoring” refer to the act of detecting, identifying and/or quantifying a target species in the atmosphere.

The term “apparent absorbance spectrum” refers to a measurement of an absorbance spectrum, i.e., a plot of absorbance units on the y-axis versus frequency (or wavelength) on the x-axis, wherein the features of the spectrum are a combination of the absorption features of the target species and the extinction of the optical beam due to scattering by particles and/or aerosols in the optical beam.

A “background spectrum” refers to a single-beam spectrum that does not contain the spectral features of the species of interest, e.g., the target species.

A “single-beam spectrum” refers to the radiant power measured by the instrument detector as a function of frequency (or wavelength). In Fourier transform infrared spectrometry, the single-beam spectrum is obtained after a fast Fourier transform of an interferogram.

A “synthetic background spectrum” refers to a background spectrum that is generated by choosing points along the envelope of a single-beam spectrum and fitting a series of short, straight lines or a polynomial function to the chosen data points to simulate the instrument response in the absence of absorbing gases or vapors.

A “point monitor” refers to a monitor that measures the concentration of a target species at a single point or location.

The term “fenceline” refers to a property line, perimeter, or outer boundary of, including, but not limited to, an industrial facility, a chemical weapons stockpile, a large area pollution source, a military base or camp, or a civilian residential area. A “fenceline” often defines the monitoring path for ORS studies.

II. Extinction of Infrared Radiation by a Particle/Gas Mixture

Low vapor pressure noxious compounds, such as toxic industrial chemicals, agricultural chemicals, e.g., pesticides, chemical warfare agents, and bioaerosols, can be released in aerosol form. Under certain conditions, the low vapor pressure compounds can be present in an aerosolized form, in a vapor phase, adsorbed on airborne particulate matter, or combinations thereof. Thus, the volume of space that these low vapor pressure compounds occupy can comprise a particle/gas mixture. The extinction of light, e.g., IR radiation, by a particle/gas mixture can be characterized by the following mathematic description.

A sample of gas comprising only particles will exhibit both absorption and scattering dictated by the particle size distribution, the compound comprising the particles, and the wavelength of light. An aerosolized compound, however, is characterized by a complex refractive index that is wavelength dependent. The wavelength dependence of the real and imaginary components of the refractive index produces the spectral shape of the scattered light, as well as the absorption features.

Equations 1a and 1b show the interdependency of the imaginary part of the refractive index on the real part and the dependence of the imaginary part on the absorption spectrum of the particle. The complex refractive index (m) is given by:
m=n−inκ (1a)
wherein n is the real part of the refractive index and

$$κ = \frac{{σ}_{p} λ}{4 π} \qquad ( 1 b )$$

wherein σₚ is the absorption coefficient of the particle as described by Beer's law (in units of m⁻¹), and λ is the wavelength of light.

The wavelength-dependent extinction coefficient, σₑ, for the particle is given by

$${σ}_{e} ( λ ) = \frac{π}{4} {\sum }_{j} {{Q}_{e} ( λ )}_{j} \cdot {N}_{j} \cdot {d}_{j}^{2} \qquad ( 2 )$$

wherein j denotes the particle size index, Qₑ(λ) is the complex refractive index dependent extinction efficiency, N is the particle number density, and d is the particle diameter. At each wavelength, the contributions of all particle sizes add to produce the total extinction due to the particles. The particles extinction contribution to the apparent infrared absorbance spectrum can be computed by multiplying the extinction coefficient by the optical pathlength, L, of the IR beam.

As shown in equation 3, Beer's law describes the extinction contribution by the gas in the sample.
A(λ)=σ_{g}(λ)·CL (3)
wherein σ_{g} is the absorption coefficient of the gas (m²/ppm) and C is the concentration of the gas in ppm. As noted previously, L is the optical pathlength of the IR beam. Thus, the total absorbance spectrum for the particle/gas mixture is given by
A(λ)=σₑ(λ)·L+σ_{g}(λ)·CL (4)
wherein, σ_{g}, L, and C are as defined immediately hereinabove.
III. Optical Remote Sensing Systems

Optical remote sensing systems can be configured to make measurements in two distinctly different monitoring modes: the passive mode and the active mode. Many ORS monitoring applications, including military applications, historically have utilized passive ORS monitors, such as passive OP-FTIR monitors, as standoff monitors, e.g., monitors that do not need powered radiation sources or mirrors to detect chemical warfare agents in the battlefield. Other monitoring applications, such as monitoring for emissions of noxious compounds from industrial sites, landfills, and chemical warfare agent stockpiles, or personnel protection in permanent and/or semi-permanent installations, neither require nor merit standoff, passive OP-IR systems.

Considerable differences exist between passive OP-IR systems and active, modulated OP-IR systems. These differences relate to different performance characteristics. For example, in passive ORS systems, distinguishing between spectral features that are due to target species in the plume and spectral features that are due to fluctuations in the ambient background radiation can be difficult. In an active, modulated OP-IR system, the system can be conditioned to reject ambient background radiation. Thus, active, modulated OP-IR systems exhibit better detection capabilities for aerosol and gas-phase particles and report less false positive detections.

Provided immediately herein below are representative configurations of OP-IR systems, including active, modulated OP-IR systems, active, unmodulated OP-IR systems, and passive OR-IR systems.

III. A. Active, Modulated Optical Remote Sensing Systems

Active, modulated optical remote sensing systems can be configured in a monostatic monitoring mode or a bistatic monitoring mode. In the monostatic configuration, the energy source and the detector are positioned at the same end of the monitoring path. A reflecting element, such as a retroreflector, is positioned at the opposite end of the monitoring point to return the optical beam to the detector. In this configuration, the optical pathlength is twice as long as the monitoring pathlength.

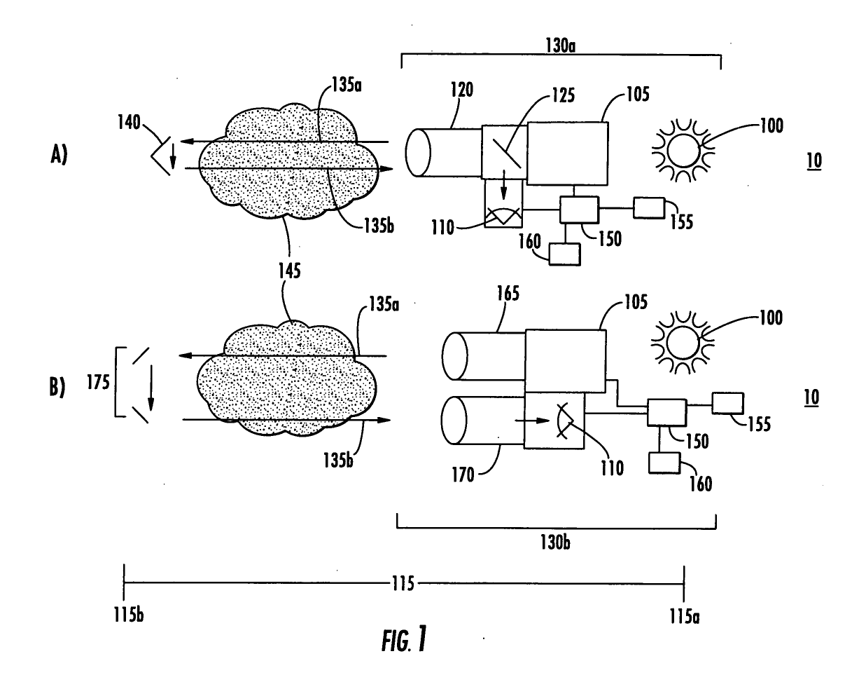

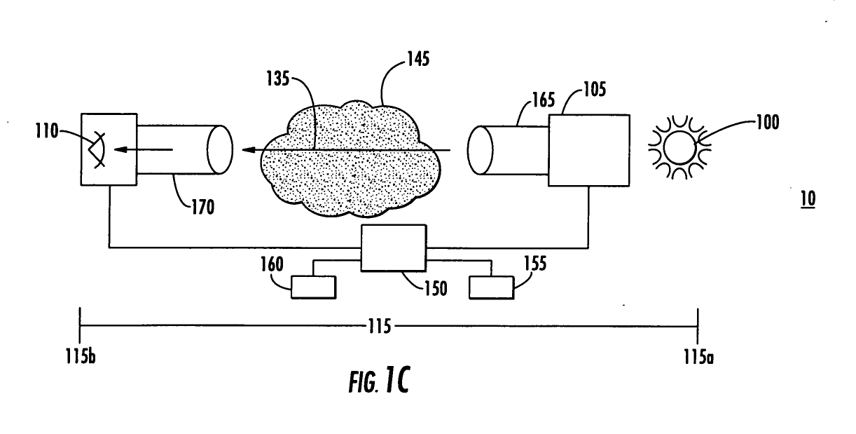

III.A.1. Active, Modulated Monostatic OP-IR Systems Schematic diagrams of two representative monostatic configurations of an OP-IR system 10 of the presently disclosed subject matter are provided in FIGS. 1A and 1B. Referring now to FIG. 1A, energy source 100, wavelength separator 105, and detector 110 are all positioned at the same end, e.g., 115a, of monitoring path 115. In this configuration, transmitting/receiving optics 120 and beamsplitter 125 also are positioned at the same end, 115a, of monitoring path 115. Thus, spectrometer module 130a comprises energy source 100, wavelength separator 105, detector 110, transmitting/receiving optics 120, and beamsplitter 125. In some embodiments, transmitting/receiving optics 120 is selected from the group consisting of a Cassegrain telescope and a Newtonian telescope.

In some embodiments, energy source 100 comprises a broadband infrared energy source, such as a globar, i.e., a silicon carbide rod, and an incandescent wire comprising nichrome or rhodium sealed in a ceramic cylinder. In such embodiments, the energy emitted by energy source 100 is modulated by, for example, an interferometer, which can comprise wavelength separator 105 or a mechanical chopper (not shown).

In some embodiments, energy source 100 comprises a pulsed broadband quantum cascade (QC) laser. The marked increased output power of a broadband cascade laser reduces the need for highly retro-reflecting mirrors and allows for the use of natural or inexpensive manmade hard targets. Thus, an OP-IR system equipped with a pulsed, broadband QC laser could be used as a compact, portable standoff detector. Accordingly, such a system can be readily moved from place to place and could be used, for example, by first responders or can be mounted on vehicles, such as emergency response vehicles, helicopters, and the like. Also, the power of the QC laser will determine the range of the instrument (up to about 500 m).

Since their first experimental demonstration in 1994, see Faist. J. et al., Science, 264, 553 (1994), QC lasers have shown remarkable progress both in terms of applications and performance. QC lasers are commercially available for all the necessary emission wavelengths, although one single laser with such broadband is most desirable for the end product aimed for the detecting and identifying aerosolized low vapor pressure compounds. A broadband QC laser with continuous emission of high power output in the 6-μm to 8-μm range (approximately 1667-cm⁻¹ to 1250-cm⁻¹ range) has been described by Gmachl et al., Nature, 415, 883-887 (2002). An extension of this laser technology into the 9-μm to 10.5-μm (approximately 1111 cm⁻¹ to 950 cm⁻¹ range) regime is possible by altering the fabrication techniques. The QC-based sensors can be used to respond to extremely low concentrations of these chemicals, before a danger is present. The pulsed mode (i.e., modulated mode) of QC laser systems will reduce any background radiation interfering with the measurements, providing the proposed active ORS system with high sensitivity, hence little false positives when compared to existing passive instruments. A holographic FTIR receiver, which has no moving mirrors for increased robustness and measurement speed, can be used with a QC laser energy source.

In some embodiments of the presently disclosed OP-IR systems, detector 110 comprises a thermal detector, such as a pyroelectric deuterated triglycine sulfate (DTGS) detector, which operates at room temperature. In some embodiments, detector 110 comprises a photoconducting detector, such as a mercury-cadmium-telluride (MCT) detector, which is cooled to liquid nitrogen temperatures.

One of ordinary skill in the art would recognize that any infrared source and any infrared detector could be used in the presently described systems. The output power of the infrared source should be stable. If the output power of the infrared source is not stable, it should be controlled. Preferably, the power fluctuations of the infrared source should be less than or on the order of the noise level of the system.

Also, the detection range of detector 110 should be matched to the spectral range of energy emitted by energy source 100. Accordingly, in some embodiments, the presently disclosed OP-IR systems comprise an energy source, detector, and other optical components, such as mirrors, beamsplitters, and the like, which are designed to operate in the mid-infrared spectral range (e.g., approximately a 2-μm to 20-μm (about 5000-cm⁻¹ to about 500-cm⁻¹) spectral range). In some embodiments, the OP-IR systems are designed to operate in the 4000-cm⁻¹ to 700-cm⁻¹ range. In some embodiments, the OP-IR systems are designed to operate in the approximately 1650-cm⁻¹ to 1250-cm⁻¹ range. In some embodiments, the OP-IR systems are designed to operate in the 1400-cm⁻¹ to 700-cm⁻¹ range. In some embodiments, the OP-IR systems are designed to operate in the 1100-cm⁻¹ to 900-cm⁻¹ range.

Referring again to FIG. 1A, the same optical device, e.g., a telescope, is used to transmit and receive optical beams 135a and 135b along monitoring path 115. To transmit and receive optical beams 135a and 135b with the same telescopic optics, e.g., transmitting/receiving optics 120, beamsplitter 125 must be positioned to divert part of returned optical beam 135b to detector 110. Thus, in this configuration, the optical beam, i.e., optical beams 135a and 135b, traverses beamsplitter 125 twice.

Referring once again to FIG. 1A, reflecting element 140 is positioned at an opposite end, e.g., 115b, of monitoring path 115. In this embodiment, reflecting element 140 comprises a single reflecting element, e.g., a cube-corner retroreflector array or a flat mirror, which returns optical beam 135 substantially along the same direction from which it was transmitted. In embodiments in which energy source 100 comprises a QC laser, reflecting element 140 can comprise a natural target.

Continuing with FIG. 1A, energy, e.g., infrared radiation, (shown as a solid arrow) is emitted from energy source 100 and directed through wavelength separator 105, e.g., an interferometer, where the energy is modulated at a predetermined frequency. In embodiments wherein the wavelength separator comprises an interferometer, the modulation frequency is wavelength dependent. The modulated energy exits wavelength separator 105, and in some embodiments, is collimated by transmitting/receiving optics 120 before it is transmitted along monitoring path 115, where it interrogates plume 145. Transmitted optical beam 135a is then redirected back toward opposite end 115b of monitoring path 115 by reflecting element 140. In some embodiments, reflecting element 140 comprises a cube-corner retroreflector array. In this configuration, reflecting element 140 returns transmitted optical beam 135a along substantially the same direction from which it came. Thus, the transmitted beam and returned beam travel along substantially the same path. Returned optical beam 135b is then collected by transmitting/receiving optics 120 and directed to detector 110 by beamsplitter 125. Detector 110 then records a signal that is indicative of the apparent absorbance spectrum of gases, vapors, aerosol, and particles comprising plume 145. Detector 110 is operatively coupled to processor 150. Processor 150 is bidirectionally coupled to memory 155, in which a plurality of machine instructions and/or data recorded by the ORS instrument are stored. Processor 150 also is operatively coupled to display/printer 160, which provides an image of the OP-IR data.

In some embodiments, OP-IR system 10 described in FIG. 1A comprises an open-path Fourier transform infrared system, in which the energy emitted from energy source 100 is modulated by wavelength separator 105, e.g., an interferometer. Thus, processor 150 can be instructed to accept only the modulated radiation from energy source 100 and to reject unmodulated ambient radiation. Accordingly, such a configuration allows the cancellation of background radiation that could introduce noise and error to the measurement due to atmospheric temperature scintillation effects.

Further, because detector 110 and wavelength separator 105 are at the same end of monitoring path 115, e.g., end 115a, the pathlength of monitoring path 115 is not limited by communication requirements between detector 110 and wavelength separator 105. For example, OP-FTIR monitors in a monostatic configuration can achieve a monitoring pathlength of about 500 m (optical pathlength of 1000 m).

Also, the monostatic configuration shown in FIG. 1A is adaptable to monitoring multiple paths in rapid succession. For example, a plurality of reflecting elements 140 can be positioned at a plurality of predetermined locations, e.g., a plurality of locations defined by a plurality of opposite ends 115b, to define a plurality of monitoring paths 115. In such a configuration, spectrometer module 130 comprising energy source 100, wavelength separator 105, detector 110, beamsplitter 125, and transmitting/receiving optics 120 can be mounted on a positioning device, such as a turntable (not shown), which allows spectrometer module 130a to be rotated in a horizontal plane, or a gimbal mechanism (not shown), which allows spectrometer module 130a to be maneuvered in three dimensions such that transmitting/receiving optics 120 direct optical beam 135 along a plurality of monitoring paths 115. Such positioning devices allow a single OP-IR spectrometer module, e.g., 130a, to be repositioned to scan a plurality of monitoring paths 115 in a horizontal plane, a vertical plane, and combinations thereof as desired. In such embodiments, the OP-IR system is referred to as a “scanning OP-IR system.” See U.S. Pat. No. 6,542,242 to Yost et al., which is incorporated herein by reference in its entirety. Alternatively, instead of employing a mechanical positioning device, optical beam 135 can be optically steered to scan a plurality of monitoring paths 115. Accordingly, scanning OP-IR monitors can be used to provide surveillance over a large area.

Referring now to FIG. 1B, and to OP-IR system 10 presented therein, and wherein like elements are identified by the same reference number as like elements in FIG. 1A, energy source 100, wavelength separator 105, transmitting optics 165, receiving optics 170, and detector 110 are each positioned at the same end, e.g., 115a, of monitoring path 115. Energy source 100, wavelength separator 105, transmitting optics 165, receiving optics 170, and detector 110 together comprise spectrometer module 130b. Reflecting element 175 is positioned at an opposite end, 115b, of monitoring path 115. In some embodiments, reflecting element 175 comprises an arrangement of mirrors, such as a single cube-corner retroreflector, that translates, e.g., shifts in a horizontal plane, transmitted optical beam 135a slightly so that is does not fold back on itself. In some embodiments, transmitting optics 165 and receiving optics 170 are each selected from the group consisting of a Cassegrain telescope and a Newtonian telescope.

Referring once again to FIG. 1B, receiving optics 170 are slightly removed from transmitting optics 165 so as to be in a position to receive returned optical beam 135b. In this configuration, detector 110 is disposed on an axis of returned optical beam 135b that is shifted in a horizontal plane relative to the axis of transmitted optical beam 135a.

In some embodiments, OP-IR system 10 described in FIG. 1B comprises an open-path Fourier transform infrared (OP-FTIR) system. Energy (shown as a solid arrow) is emitted from energy source 100 and directed through wavelength separator 105, e.g., an interferometer, where the energy is modulated at a predetermined frequency. In embodiments wherein the wavelength separator comprises an interferometer, the modulation frequency is wavelength dependent. The modulated energy exits wavelength separator 105, and in some embodiments, is collimated by transmitting optics 165 before it is transmitted along monitoring path 115, where it interrogates plume 145. Transmitted optical beam 135a is then redirected back toward the opposite end, 115a, of monitoring path 115 by reflecting element 175. In some embodiments, reflecting element 175 comprises a single cube-corner retroreflector. As shown in FIG. 1B, reflecting element 175 translates returned optical beam 135b such that returned optical beam 135b and transmitted optical beam 135a are no longer traveling along identical paths. Returned optical beam 135b is then collected by receiving optics 170, then focused onto detector 110, which records a signal that is indicative of the apparent absorbance spectrum of gases, vapors, aerosol, and particles comprising plume 145.

Detector 110 is operatively coupled to processor 150. Processor 150 is bidirectionally coupled to memory 155, in which a plurality of machine instructions and/or data recorded by the ORS instrument are stored. Processor 150 also is operatively coupled to display/printer 160, which provides an image of the OP-IR data.

Because initial alignment with this configuration can be difficult, this type of monostatic ORS system typically is used in permanent installations rather than as a transportable unit.

III.A.2. Active. Modulated Bistatic OP-IR Systems

In a bistatic configuration, the detector and the energy source are at opposite ends of the monitoring path. In this case, the optical pathlength is equal to the monitoring pathlength. In one bistatic configuration, the energy source, wavelength separator, e.g., an interferometer, and transmitting optics are positioned at one end of the monitoring path and the receiving optics and detector are positioned at the opposite end of the monitoring path.

Referring now to FIG. 1C, a schematic diagram of an active, modulated bistatic OP-IR system 10 is presented, and like elements are identified by the same reference number as like elements in FIGS. 1A and 1B. Energy source 100, wavelength separator 105, and transmitting optics 165 are positioned at one end, 115a, of monitoring path 115 and receiving optics 170 and detector 110 are positioned at an opposite end, 115b, of monitoring path 115. Receiving optics 170 can comprise an optical telescope or other optical device that defines the field of view of the instrument.

Detector 110 is operatively coupled to processor 150. Processor 150 is bidirectionally coupled to memory 155, in which a plurality of machine instructions and/or data recorded by the ORS instrument are stored. Processor 150 also is operatively coupled to display/printer 160, which provides an image of the OP-IR data.

Referring once again to FIG. 1C, energy, e.g., infrared radiation, (shown as a solid arrow) is emitted from energy source 100 and directed through wavelength separator 105, e.g., an interferometer, where the energy is modulated at a predetermined frequency. In embodiments wherein the wavelength separator comprises an interferometer, the modulation frequency is wavelength dependent. The modulated energy exits wavelength separator 105, and in some embodiments, is collimated by transmitting optics 165 before it is transmitted along monitoring path 115, where it interrogates plume 145. Plume 145 can comprise a mixture of noxious compounds, wherein the noxious compounds can be in a gas phase, vapor phase, aerosol phase, adsorbed on airborne particulate matter, and combinations thereof, airborne particulate matter, and atmospheric gases. Optical beam 135 is then collected by receiving optics 170, then focused on detector 110, which records a signal that is indicative of the apparent absorbance spectrum of gases, vapors, aerosols, and particles comprising plume 145.

An advantage of the bistatic configuration shown in FIG. 1C is that optical beam 135 is modulated before it is transmitted along monitoring path 115. Processor 150 can be instructed to accept only the modulated radiation from the energy source and to reject unmodulated extraneous radiation, such as ambient or background radiation. Accordingly, such a configuration allows the cancellation of ambient or background radiation that could introduce noise and error to the measurement due to atmospheric temperature scintillation effects.

The maximum distance that wavelength separator 105 and detector 110 can be separated should be established with care, however, because communication between detector 110 and wavelength separator 105, e.g., an interferometer, is required for timing purposes during the acquisition of the spectrum. For example, a bistatic OP-FTIR system with this configuration developed for monitoring workplace environments had a maximum monitoring pathlength of about 40 m. See Xiao, H. K., et al., Am. Ind. Hyg. Assoc. J., 52, 449 (1991).

III.B. Unmodulated Optical Remote Sensing Systems

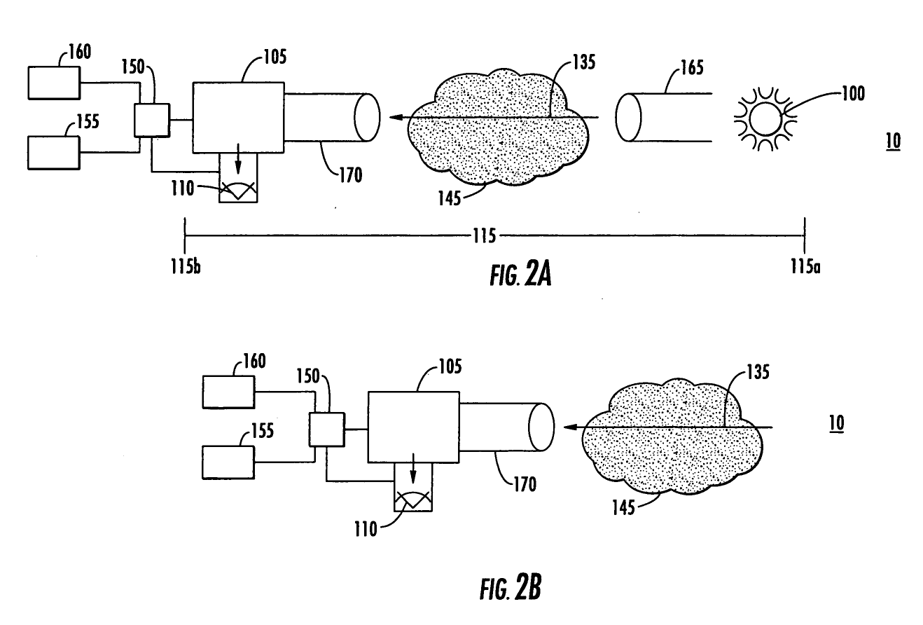

Unmodulated optical remote sensing systems can acquire spectral data in an active mode or a passive mode. FIGS. 2A and 2B show representative configurations of unmodulated OP-IR systems.

III.B.1. Active, Unmodulated Bistatic OP-IR Systems

Referring now to FIG. 2A, another embodiment of OP-IR system 10 is presented, and like elements are identified by the same reference number as like elements in FIGS. 1A-1C. Energy source 100 and transmitting optics 165 are positioned at one end, e.g., 115a, of monitoring path 115 and receiving optics 170, wavelength separator 105, and detector 110 are positioned at the opposite end, e.g., 115b, of monitoring path 115. In this configuration, transmitting optics 165 typically comprise a paraboloid-shaped mirror, or other suitable collimating device, which collimates optical beam 135 before it is transmitted along monitoring path 115.

Referring once again to FIG. 2A, energy, e.g., infrared radiation, (shown as a solid arrow) is emitted from energy source 100 and is collimated by transmitting optics 170 before it is transmitted along monitoring path 115, where it interrogates plume 145. Optical beam 135 is then collected by receiving optics 170, directed through wavelength separator 105, and then focused on detector 110, which records a signal that is indicative of the apparent absorbance spectrum of gases, vapors, aerosol, and particles comprising plume 145.

A consideration to the bistatic configuration shown in FIG. 2A is that the energy from energy source 100 is not modulated before it is transmitted along monitoring path 115. Therefore, energy emitted by energy source 100 and energy from the ambient background in the field of view of receiving optics 170 can be difficult to distinguish by electronic processing.

Another consideration to bistatic systems in general is that if multiple paths are to be monitored in rapid succession, e.g., by monitoring along different paths near different fencelines of an industrial facility, multiple sources or multiple detectors, or a combination of multiple sources and multiple detectors are required. This requirement can result in additional expense and complexity to the monitoring scheme.

III.B.2. Passive Optical Remote Sensing Systems

In contrast to the active ORS systems described hereinabove, a passive ORS system comprises a configuration that is similar to the bistatic configuration shown in FIG. 2A, except that the passive ORS system relies on ambient background radiation, which is emitted from natural surfaces that are only a few degrees different in temperature from the absorbing or emitting medium as the energy source.

Referring now to FIG. 2B, wherein like elements are identified by the same reference number as like elements in FIGS. 1A-1C and 2A, passive OR-IR system 10 comprises only the following optical components: receiving optics 170, wavelength separator 105, and detector 110. If the temperature of plume 145 is higher than the temperature of the ambient background in the field of view of receiving optics 170, the species comprising plume 145 will exhibit emission lines. If the temperature of the ambient background in the field of view of receiving optics 170 is higher than that of plume 145, the species comprising plume 145 will attenuate the radiation emitted by the ambient background and thus produce absorption lines.

Because it can be difficult to distinguish between spectral features that are due to target species in the plume and spectral features that are due to fluctuations in the ambient background radiation, passive OP-IR systems are of limited utility for detecting, identifying, and quantifying low vapor pressure noxious compounds in the atmosphere.

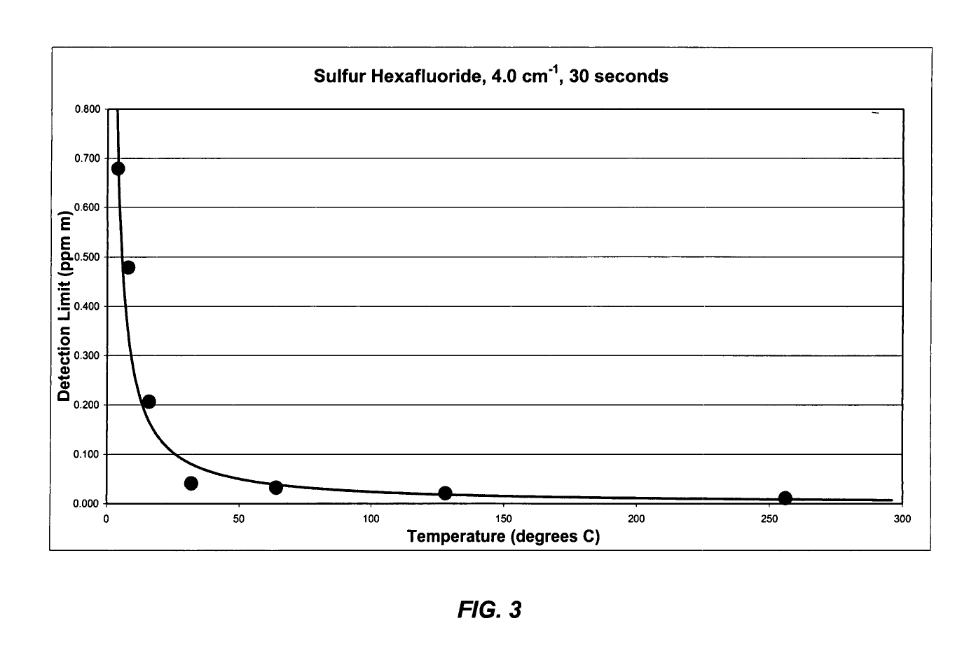

Further, a typical active OP-IR monitor utilizes an infrared source, which operates at a temperature ranging from about 300K to about 1500 K, and which can be either modulated or unmodulated. Referring now to FIG. 3, the relationship between detection limits of an OP-IR system and the operating temperature of the radiation source is shown. As shown in FIG. 3, detection levels for sulfur hexafluoride were determined for source temperatures ranging from about 4° C. to about 300° C. above ambient conditions for a non-modulated bistatic configuration. These results indicate that as little as 70° C. above ambient is sufficient to achieve a marked improvement in detection limits for an active OP-IR system as compared to the passive OP-IR approach.

The high source temperature of an active OP-IR system can provide more than an 80-fold increase in the infrared radiant flux emitted per unit area in the 7-14-μm spectral fingerprint region compared to passive OP-IR systems. As a result, active OP-IR monitors can detect chemical warfare agents, such as, but not limited to GA, GB, GD, HD and Lewisite in the range of 1 to 10 μg/m³ or below. These detection limits are orders of magnitude lower than those obtainable by passive OP-IR systems.

For example, the estimated detection limits of OP-IR methods for detecting representative chemical warfare agents (CWAs) in the vapor phase are compared to point source monitoring methods in Table 1.

TABLE 1
Estimated Monitoring Ranges for Representative Chemical Agents
in the Vapor Phase

| | Active | Passive | | | | |
|---|---|---|---|---|---|---|
| Chemical | OP-IR | OP-IR | NRT | DAAMS | IDLH | AEGL |
| Agent | (μg/m³) | (μg/m³) | (μg/m³) | (μg/m³) | (μg/m³) | (μg/m³) |
| GB | 1 × 10⁻⁴ to 1 | 10 to 80 | 2.5 × 10⁻⁵ to | 5 × 10⁻⁷ to 5 × 10⁻⁴ | 5 × 10⁻² | 5 × 10⁻² |
| | | | 4.5 × 10⁻³ | | | |
| VX | 1 × 10⁻⁴ to 1 | 10 to 80 | 2.5 × 10⁻⁶ to | 5 × 10⁻⁷ to 5 × 10⁻⁵ | 8 × 10⁻³ | 6 × 10⁻³ |
| | | | 5 × 10⁻³ | | | |
| HD | 1 × 10⁻⁴ to 1 | 10 to 80 | 1 × 10⁻⁴ to | 2 × 10⁻⁵ to 7 × 10⁻⁴ | na | 1 × 10⁻¹ |
| | | | 2 × 10⁻² | | | |

NRT = near real-time;
DAAMS = depot area air monitoring system;
IDLH = Immediately Dangerous to Life and Health;
AEGL = Acute Exposure Guideline Level
na = not available

As shown in Table 1, OP-IR methods are capable of detecting representative CWAs in the vapor phase at levels well below the Immediately Dangerous to Life and Health (IDLH) and Acute Exposure Guideline Level (AEGL) limits for these CWAs.

The wide range of values shown in Table 1 depends on many measurement variables, such as source temperature, source modulation, type of detector, type of infrared source (for example, QC lasers could provide unprecedented low detection limits), pathlength through the plume relative to the optical path length, atmospheric conditions, and the like. Yet, for each specific measurement-and-system condition, the detection limit can be accurately determined, thereby screening out unwanted false positive readings. This feature allows the users to exploit the benefits of path-integrated measurements, i.e., better capture of the entire plume, and still make use of several complementary sensitive point monitors for detection confirmation. These point monitors by themselves (without path-integrated data) can bias—most typically by underestimation of the extent of the plume—or worse, miss the entire plume. When multiple beams are scanned in different directions and path-lengths, a radial plume mapping (RPM) method can be applied to retrieve spatial gradients and profiles across the plume. Such systems can detect more than 100 noxious compounds, such as but not limited to, TICs and/or CWAs, simultaneously.

IV. Active. Modulated Open-Path Infrared Method, System, and Computer Program Product for Detecting, Identifying, and Quantifying One or More Low Vapor Pressure Noxious Compounds in the Atmosphere

The presently disclosed subject matter provides an active, modulated open-path infrared (OP-IR) method, system, and computer program product for monitoring one or more low vapor pressure noxious compounds in the atmosphere. The presently disclosed method is capable of detecting, identifying, and quantifying low vapor pressure compounds in the vapor phase, aerosolized phase, when adsorbed on airborne particulate matter, and combinations thereof.

In some embodiments, the method of monitoring one or more low vapor phase noxious compounds in the atmosphere comprises providing an active OP-IR system, wherein the active OP-IR system comprises a modulated energy source. Any of the active OP-FTIR systems shown in FIGS. 1A-1C, in which the energy source is modulated, are suitable for the presently disclosed methods. In some embodiments, the OP-IR system comprises an active, monostatic OP-IR system as shown in FIG. 1A. One of ordinary skill in the art would recognize that the presently disclosed subject matter, however, is not limited to embodiments shown in FIGS. 1A-1C.

To monitor for low vapor pressure noxious compounds in the atmosphere using an OP-IR system, a monitoring path is first selected. The monitoring path can be selected to run parallel, for example, to the fenceline of an industrial facility or a chemical weapons stockpile, along which low vapor pressure noxious compounds emitted from the industrial facility or chemical weapons stockpile are to be measured. In such embodiments, a plume comprising the one or more low vapor pressure noxious compounds can pass across the monitoring path through a variety of mechanisms, including diffusion in the air, dispersion by prevailing wind currents, and the like.

The monitoring path also can be positioned near the perimeter of, for example, a civilian residential area or a military base or camp, along which the potential release of low vapor pressure noxious compounds is monitored to provide an early warning to the civilians or military personnel housed therein. The monitoring path also can be positioned downwind, for example, from a pesticide release in an open field to monitor for low vapor pressure compounds comprising a plume resulting from pesticide drift. Guidelines for selecting a monitoring path are provided in ASTM E 1865-97 Standard Guide for Open-Path Fourier Transform Infrared (OP/FT-IR) Monitoring of Gases and Vapors in Air and ASTM E 1982-98, Standard Practice for Open-Path Fourier Transform Infrared (OP/FT-IR) Monitoring of Gases and Vapors in Air, both of which are incorporated herein by reference in their entirety.

Once the monitoring path is selected, spectrometer module 130a, as shown in FIG. 1A, is positioned along a line of measurement, such that the position of spectrometer module 130a defines one end, e.g., 115a, of monitoring path 115. Reflecting element 140, e.g., a cube-corner retroreflector array, also is positioned along the line of measurement, at a predetermined distance from spectrometer module 130a, such that the position of reflecting element 140 defines an end, e.g. 115b, opposite that of end 115a of monitoring path 115. Ends 115a and 115b of monitoring path 115 should be selected so that they capture the expected plume, e.g., plume 145, of low vapor pressure noxious compounds.

Once the OP-IR system is set-up along the line of measurement, the instrumental operating parameters are selected. Guidelines for selecting operating parameters for OP-IR systems are provided in ASTM E 1865-97 Standard Guide for Open-Path Fourier Transform Infrared (OP/FT-IR) Monitoring of Gases and Vapors in Air and ASTM E 1982-98, Standard Practice for Open-Path Fourier Transform Infrared (OP/FT-IR) Monitoring of Gases and Vapors in Air.

Prior to monitoring for low vapor pressure noxious compounds of interest, a background spectrum is recorded. The background spectrum should not contain any spectral features of the low vapor pressure noxious compounds of interest. Further, the background spectrum should not produce a baseline offset in the measured apparent absorbance spectrum. Thus, in some embodiments, the background spectrum is recorded along the same monitoring path, with the same instrumental configuration over which the low vapor pressure noxious compounds are to be monitored. A background spectrum can be selected from a plurality of spectra, e.g., a time series of spectra, acquired along the monitoring path during a monitoring period in which low vapor pressure noxious compounds are not present in the path. Guidelines for generating and selecting a background spectrum are provided in ASTM E 1865-97 Standard Guide for Open-Path Fourier Transform Infrared (OP/FT-IR) Monitoring of Gases and Vapors in Air and ASTM E 1982-98, Standard Practice for Open-Path Fourier Transform Infrared (OP/FT-IR) Monitoring of Gases and Vapors in Air.

Once a suitable background spectrum is generated, OP-IR spectra are recorded along the path at predetermined time intervals. Each low vapor pressure noxious compound exhibits characteristic and unique features in an infrared (IR) spectrum, i.e., a “molecular fingerprint,” which can be measured and exploited for identification purposes. The location and shape of these characteristic and unique features in the infrared spectrum depend on the identity of the low vapor pressure noxious compound and the physical state, e.g., vapor phase, aerosol, adsorbed on particle, in which it exists.

The apparent absorbance spectrum of aerosolized or particle bound low vapor pressure compounds also exhibits certain characteristics, such as but not limited to a wavelength dependent baseline offset, which is indicative of the presence of an aerosol plume in the optical beam and the typical aerosol size. Fine aerosols exhibit a stronger extinction contribution to the apparent absorbance spectrum at the higher frequency (shorter wavelength) end of the spectrum, which results in the appearance of slightly negative slope in the baseline offset.

Further, absorption by aerosol material (liquid or solid), as expressed by the imaginary part of the complex refractive index, occurs at particular wavelengths for a particular aerosolized material, typically in the fingerprint region of the infrared spectral range, e.g., from about 1400 cm⁻¹ to about 900 cm⁻¹. In the extinction spectrum of a suspended aerosol cloud, this absorption usually will be represented by a derivative-like shear of the spectrum baseline if size-dependent scattering occurs in the region. The width of the absorption features of an aerosolized low vapor pressure noxious compound can vary slightly with a change in particle size. If the particles are very small or several absorption lines are adjacent to each other, the absorption feature in the extinction spectrum might look similar to typical gas absorption features (e.g., Lorentzian line shape). Regardless of the shape, the magnitude of the absorption feature, i.e., an absorption band and/or a derivative-like feature, correlate with the scattering baseline offset, as both phenomena indicate the presence of an aerosol cloud in the optical beam.

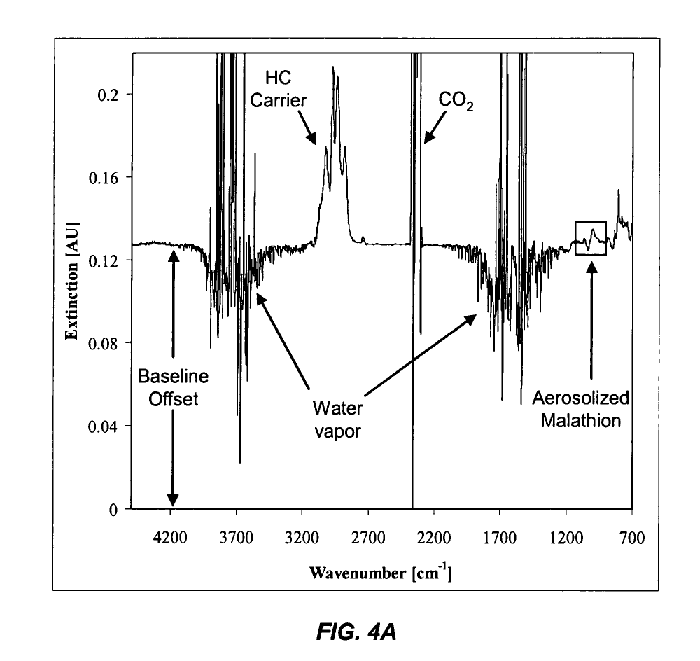

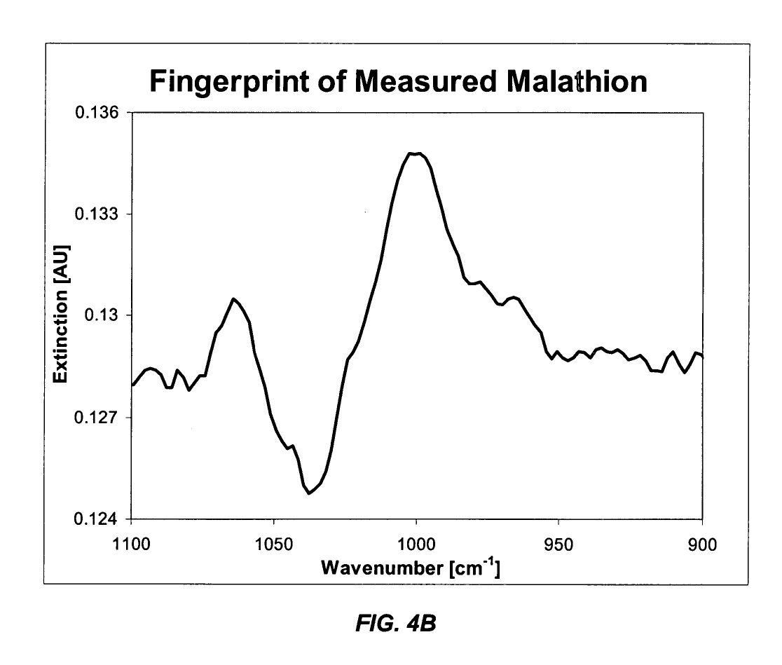

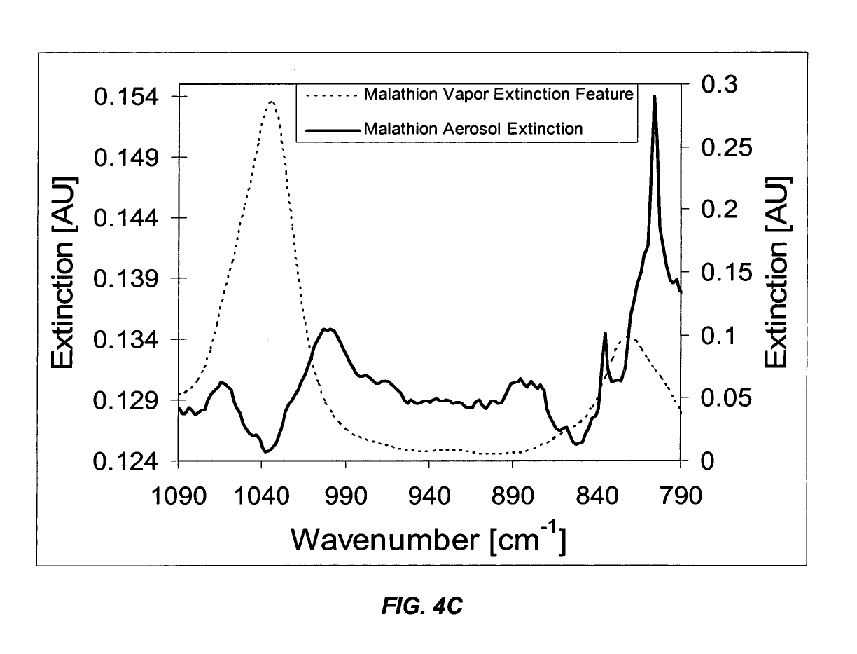

An example of an OP-IR spectrum of an aerosolized low vapor pressure noxious compound is provided in FIGS. 4A and 4B, which show an OP-IR spectrum of aerosolized malathion. Referring now to FIG. 4A, the OP-IR spectrum of aerosolized malathion exhibits a wavelength dependent baseline offset, which provides information about the presence of an aerosol plume in the optical beam and the typical aerosol size. As shown in FIG. 4A, the fine pesticide aerosol exhibits a stronger extinction contribution at the higher frequency (shorter wavelength) end of the spectrum, which results in the appearance of slightly negative slope in the baseline offset. In this example, the malathion also employed a hydrocarbon carrier, which exhibits a C—H stretching mode in the 3000-cm⁻¹ spectral region.

Referring once again to FIG. 4A, the apparent absorbance spectrum of aerosolized malathion exhibits a representative characteristic, namely derivative-like features in the fingerprint region of the infrared spectrum, e.g., from 1400 to 700 cm⁻¹. FIG. 4B shows the spectrum shown in FIG. 4A expanded in the 1100- to 900-cm⁻¹ spectral region. These features are a result of the interdependence between the imaginary and real parts of the complex refractive index in the vicinity of an absorption feature of the aerosolized material. The specificity of this feature, e.g., the location and the shape of this feature, facilitates the identification of low vapor pressure noxious compounds. This feature is unique for each compound. A positive identification can be made, for example, by comparing the measured apparent absorbance spectrum with reference infrared spectra of known low vapor pressure noxious compounds. This comparison can be done manually or can be automated to provide a real-time or near real-time identification of one or more low vapor pressure noxious compounds in the monitoring path. Also, the magnitude of this unique feature correlates well with the rise in the baseline offset, which makes possible the verification of the presence of a low vapor pressure compound in the aerosol or particle phase.

Further, at high concentrations of low vapor pressure noxious compounds, as would be expected in circumstances involving hazardous exposure levels, spectral features due to the low vapor pressure compound in the vapor phase would likely be observed in addition to the spectral features of the aerosolized low vapor pressure compound. The OP-IR spectrum of aerosolized malathion shown in FIGS. 4A and 4B does not exhibit absorption features due to vapor phase malathion. Referring now to FIG. 4C, a spectrum of vapor phase malathion, which exhibits characteristic absorption bands of malathion in the mid-infrared spectral range, is compared to the apparent absorbance spectrum of aerosolized malathion. The presence of these absorption bands in the measured apparent absorbance spectrum would indicate that the low vapor pressure noxious compound is present in the vapor phase along the monitoring path. The magnitude of these absorption bands correlate with the path-integrated concentration of the low vapor pressure noxious compound in the monitoring path. The presence of such absorption bands, which can be described as a characteristic of the apparent absorbance spectrum, in addition to a baseline offset and derivative-like features would indicate that the low vapor pressure noxious compound is present in both the vapor phase and an aerosolized or particle phase.

Also, the presently disclosed method also can be used to determine the relationship between carrier concentration, which is likely to be present in the vapor phase, and active ingredient concentration, such as but not limited to, malathion.

Low vapor pressure organophosphate pesticides, such as malathion, are similar to organophosphate chemical warfare agents, such as, but not limited to, VX, GA, and GF, both in their infrared absorption characteristics and volatility. For example, the vapor pressure of GA, GF, and VX at 20° C. is 0.037 Torr, 0.044 Torr, and 0.0007 Torr, respectively.

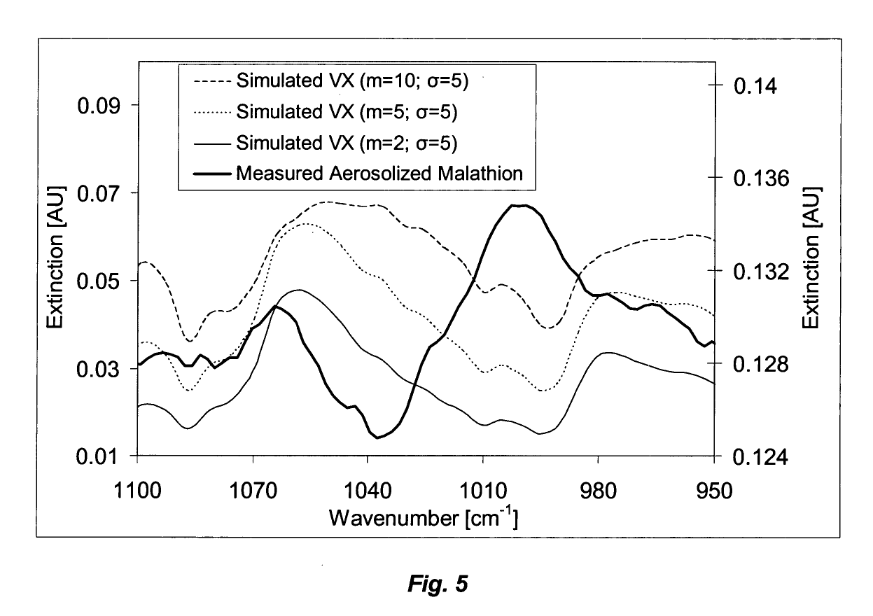

Referring now to FIG. 5, in a simulation experiment, a VX aerosol extinction spectrum was calculated for the 950-1100 cm⁻¹ (9.1-10.5 μm) spectral region, using the known complex refractive index spectrum of VX in this spectral region. See Flanigan. D. F., “The Spectral Signatures of Chemical Agent Vapors and Aerosols,” CRDEC-TR-85002, (Clearinghouse for Federal Scientific and Technical Information, Cameron Station, Va., 1985).

Three extreme size distribution scenarios (normal distribution with mean distribution of m and standard deviation of sigma) are shown in FIG. 5. The derivative-like features of the simulated VX spectrum shown in FIG. 5 are similar to the measured features of aerosolized malathion (thick solid line), except that two derivative-like features are observed in the 1100-950 cm⁻¹ region for VX, whereas one feature is observed between 1040-1000 cm⁻¹ for malathion (see also FIG. 4B). Thus, as shown in FIG. 5, VX can be distinguished from malathion by the location and shape of the derivative-like features in the fingerprint region.

The rising side of the feature (or shear) is specific to each type of material (e.g., aerosolized low vapor pressure compound or low vapor pressure compound adsorbed on airborne particulate matter) in the specific spectral region in which it appears. Because VX has two of these features in this region, the probability of detection and identification is very high with a minimal likelihood of a false positive and false negative identification.

The specific region of the shear is independent of size distribution for a large range of mean aerosol size, although the baseline offset and feature's shape can vary with the amount of aerosol and the size distribution of particles present in the optical beam.

Assuming similar optical absorption and density properties for the two compounds, the minimum detection level for VX over a 200-m pathlength is estimated to be approximately 50 μg/m³ in the liquid phase with a data collection time (integration) of two seconds. At such concentrations, gas phase features are not expected to be observed. In more acute exposure levels, however, the gas phase can be detected, and support the identification of the chemical agent. These types of very sensitive identification capabilities are most feasible with an active, modulated monostatic OP-IR system.

Thus, FIG. 5 demonstrates that the features mentioned above can be used for successful identification of VX from spectra acquired in the IR region ranging from 1100 to 950 cm⁻¹ (9.1-10.5 μm). Simulations of monodisperse VX aerosol lead to the same conclusion.

In addition to measuring the apparent absorbance spectrum of aerosolized low vapor pressure compounds, the presently disclosed method can be used to measure the apparent absorbance spectra of low vapor pressure compounds that are adsorbed on airborne particulate matter. The apparent absorbance spectrum of a low vapor pressure compound adsorbed on airborne particulate matter, such as a dust particle, also exhibit a derivative-like feature similar to that in the apparent absorbance spectrum of an aerosolized low vapor pressure compound in the fingerprint region of the infrared spectrum. Such spectra exhibit a monotonic increase in the specificity of the derivative-like feature with increasing amount of the low vapor pressure compound adsorbed on the particulate matter. The higher the specificity of this derivative-type feature for a particular low vapor pressure compound, the less likelihood of a false positive or false negative identification. For example, because the chemical agent VX has two of these derivative-type features in this region, the probability of detection and identification is very high with a minimal likelihood of a false positive and false negative identification. The specific region of the shear is independent of size distribution for a large range of mean aerosol size, although the baseline offset and feature's shape can vary with the amount of aerosol and the size distribution of particles present in the optical beam.

The apparent absorbance spectrum of a low vapor pressure compound adsorbed on airborne particulate matter also exhibits extinction features of the airborne particulate matter. The presence of both the derivative-type features and the extinction features provides the ability of ORS techniques to remotely identify low vapor pressure compounds in the liquid aerosolized phase as well as low vapor pressure compounds adsorbed on airborne particulate matter.

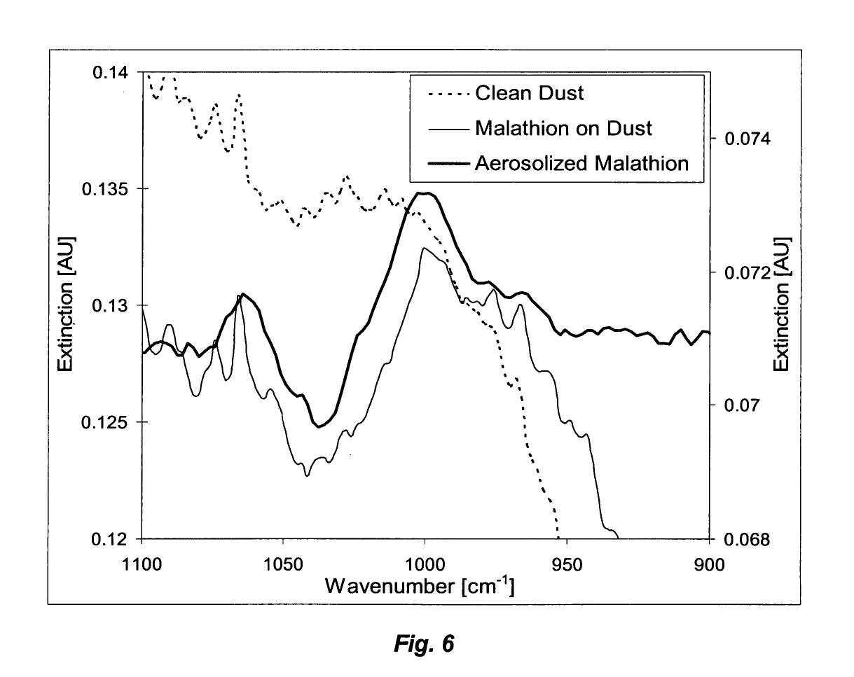

For example, FIG. 6 shows OP-IR spectra of a plume of dust particles and plumes comprising mixtures of different amounts of malathion, either in an aerosolized form or adsorbed on the airborne dust particles. Referring now to FIG. 6, the thick solid line shows the apparent absorbance spectrum of aerosolized malathion in the absence of dust particles; the thin solid line shows the apparent absorbance spectrum of malathion adsorbed on airborne dust particles; and the dotted line shows the apparent absorbance spectrum of airborne dust particles.

Continuing with FIG. 6, the apparent absorbance spectrum of malathion adsorbed on airborne dust particles (thin solid line) exhibits a derivative-like feature similar to aerosolized malathion in the 950- to 1100-cm⁻¹ region (thick solid line) and dust extinction features (dotted line). The presence of both features in this spectrum of malathion adsorbed on airborne dust particles demonstrates the capabilities of the presently disclose ORS method to remotely identify low vapor pressure compounds in the liquid aerosolized phase as well as low vapor pressure compounds adsorbed on airborne particulate matter. This behavior was observed in each of the different mixtures of malathion adsorbed on dust particles. The apparent absorbance spectrum of these mixtures exhibited a monotonic increase in the specificity of the derivative-like feature with increasing amount of malathion adsorbed on the dust particles.

Thus, the presently disclosed subject matter is directly applicable to detecting and identifying of such low-vapor pressure noxious compounds in a plume, including, but not limited to, a plume of aerosolized pesticides generated during spray field operations and related pesticide drifts; a plume of toxic industrial chemicals emitted from an industrial facility; and a plume of chemical warfare agents and/or bioaerosols released in the battlefield or toward a civilian target. In such applications, the plume can be comprised of a mixture of aerosols and gases.

Accordingly, in the presently disclosed method, the presence of a baseline offset in an OP-IR spectrum recorded along a monitoring path indicates the presence of an aerosol, e.g., a liquid droplet, and/or solid particles, in the optical beam.

Further, the location and shape of one or more derivative-like features in the OP-IR spectrum can be used to identify the one or more low vapor pressure noxious compounds in monitoring path. The magnitude of the derivative-like feature can be compared to a concentration calibration curve to determine the concentration of the low vapor pressure noxious compound. A correlation of the magnitude of the derivative-like feature with the baseline offset indicates that the low vapor pressure noxious compound is present in the aerosol or particle phase. The presence of absorption bands indicates that the low vapor pressure compound is present in the vapor phase.

Thus, in some embodiments, the method for detecting a low vapor pressure compound in the atmosphere comprises:
(a) providing an instrument adapted for emitting modulated infrared radiation along a monitoring path;
(b) providing at least one detector disposed so as to detect the modulated infrared radiation emitted by the instrument, wherein the detector is capable of producing a signal indicative of the apparent absorption spectrum of the low vapor pressure compound;
(c) positioning the instrument such that the emitted modulated infrared radiation traverses the monitoring path;
(d) measuring the apparent absorption spectrum of the low vapor pressure compound, wherein the apparent absorption spectrum exhibits two or more characteristics selected from the group consisting of:
   (i) one or more absorption bands;
   (ii) one or more derivative-like features;
   (iii) one or more wavelength dependent baseline offsets; and
   (iv) combinations thereof; and
(e) correlating the two or more characteristics to provide one of:
   (i) a detection;
   (ii) an identification;
   (iii) a quantification; and
   (iv) combinations thereof;
of one or more low vapor pressure compounds to monitor the one or more low vapor pressure compounds in the atmosphere.

In some embodiments, the low vapor pressure compound comprises a physical state, wherein the physical state is selected from the group consisting of a vapor phase, an aerosol phase, adsorbed on airborne particulate matter, and combinations thereof. In some embodiments, the low vapor pressure compound comprises a toxic chemical. In some embodiments, the toxic chemical is selected from the group consisting of an industrial toxic chemical, an agricultural chemical, a chemical warfare agent, and a bioaerosol. In some embodiments, the toxic chemical comprises an organophosphate toxic chemical.

In some embodiments, the instrument comprises an active open-path Fourier transform infrared (OP-IR) spectrometer system. In some embodiments, the open-path infrared spectrometer system comprises an open-path Fourier transform infrared spectrometer system. In some embodiments, the open-path Fourier transform infrared spectrometer system comprises a monostatic configuration.

In some embodiments, the instrument comprises a pulsed quantum cascade (QC) laser infrared radiation source. In some embodiments, the instrument has a spectral range of at about 700 cm⁻¹ to about 5000 cm⁻¹. In some embodiments, the detector is selected from the group consisting of a photoconducting detector, such as a mercury-cadmium-telluride (MCT) detector and a thermal detector, such as a deuterated tryglycine sulfate (DTGS) detector.

In some embodiments, the monitoring path is positioned along a perimeter of a facility. In some embodiments, the facility is a facility having one or more toxic chemicals disposed therein. In some embodiments, the facility houses human occupants.

In some embodiments, the one or more absorption bands indicates the presence of one or more low vapor pressure compounds in a vapor phase in the monitoring path. In some embodiments, the one or more derivative-like features indicates the presence of one or more low vapor pressure compounds in one of an aerosol phase, a particle phase, and combinations thereof in the monitoring path. In some embodiments, the one or more wavelength dependent baseline offsets indicates the presence of one or more low vapor pressure compound in one of an aerosol phase, a particle phase, and combinations thereof in the monitoring path. In some embodiments, the correlating of the two or more characteristics (e.g., the one or more absorption bands, the one or more derivative-like features, and the one or more wavelength dependent baseline offsets) indicates the presence of one or more low vapor pressure compound in one of a vapor phase, an aerosol phase, a particle phase, and combinations thereof in the monitoring path.

In some embodiments, the correlating of the two or more characteristics (e.g., the one or more absorption bands, the one or more derivative-like features, the one or more wavelength dependent baseline offsets, and combinations thereof) is performed in real-time.

In some embodiments, the presently disclosed subject matter provides a system for monitoring for one or more low vapor pressure compounds in the atmosphere, the system comprising:
(a) an instrument adapted for emitting modulated infrared radiation along a monitoring path;
(b) at least one detector disposed so as to detect the modulated infrared radiation emitted by the instrument, wherein the detector is capable of producing a signal indicative of the apparent absorption spectrum of the low vapor pressure compound, and wherein the apparent absorption spectrum exhibits two or more characteristics selected from the group consisting of:
   (i) one or more absorption bands;
   (ii) one or more derivative-like features;
   (iii) one or more wavelength dependent baseline offset; and
   (iv) combinations thereof;
(c) a memory in which a plurality of machine instructions are stored; and
(d) at least one processor that is coupled to the at least one detector and the memory, wherein the processor is capable of executing the plurality of machine instructions stored in the memory, causing the processor to:
   (i) record the signal indicative of the apparent absorption spectrum of the low vapor pressure compound, wherein the apparent absorption spectrum exhibits two or more characteristics selected from the group consisting of one or more absorption bands, one or more derivative-like features; one or more wavelength dependent baseline offsets; and combinations thereof; and
   (ii) correlate the two or more characteristics (e.g., the one or more absorption bands, the one or more derivative-like features, the one or more wavelength dependent baseline offsets, and combinations thereof to provide one of a detection; an identification; a quantification; and combinations thereof of one or more low vapor pressure compounds to monitor one or more low vapor pressure compounds in the atmosphere.

In some embodiments, the instrument comprises an active open-path Fourier transform infrared (OP-FTIR) spectrometer system. In some embodiments, the open-path Fourier transform infrared spectrometer system comprises a monostatic configuration. In some embodiments, the instrument comprises a pulsed quantum cascade (QC) laser infrared radiation source. In some embodiments, the instrument has a spectral range of at about 700 cm⁻¹ to about 5000 cm⁻¹. In some embodiments, the detector is selected from the group consisting of a photoconducting detector and a thermal detector. In some embodiments, the instrument is transportable.

In some embodiments, the presently disclose subject matter provides a computer program product comprising computer-executable instructions embodied in a computer-readable medium for performing steps comprising:
(a) inputting a signal indicative of the apparent absorption spectrum of a low vapor pressure compound, wherein the apparent absorption spectrum exhibits two or more characteristics selected from the group consisting of one or more absorption bands, one or more derivative-like features, one or more wavelength dependent baseline offset, and combinations thereof; and
(b) correlating the two or more characteristics (e.g., one or more absorption bands, the one or more derivative-like features, the one or more wavelength dependent baseline offsets, and combinations thereof) to monitor for one or more low vapor pressure compounds in the atmosphere.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

Representative open-path Fourier transform infrared (OP-FTIR) spectra were recorded with an Industrial Monitor and Control Corporation (Round Rock, Tex., United States of America) OP-FTIR system with a 70-m pathlength and a two second data collection, i.e., integration, time.

A compressor and paint sprayer were used to aerosolize representative low vapor pressure compounds, for example malathion pesticide (1,2-di(ethoxycarbonyl)ethyl O,O-dimethyl phosphorodithioate). In this example, the malathion pesticide comprised a hydrocarbon carrier and a composition of about 50% carrier and 50% active ingredient, e.g, malathion. Malathion is a low vapor pressure organophosphate very similar to VX chemical agent (methylphosphonothioic acid) both in its infrared characteristics and in its volatility.

A small aerosol cloud of malathion pesticide was dispersed into the beam and the OP-FTIR spectrum was recorded. A representative spectrum of aerosolized malathion is shown in FIG. 4A. Note that a larger concentration would be expected for hazardous exposure levels, and, in those circumstances, spectral features due to vapor phase malathion would likely be observed in addition to the aerosol features. For comparison, a reference spectrum of malathion in the vapor phase is shown in FIG. 4C.

There are two aspects to FIG. 4A. The first aspect is the wavelength dependent baseline offset, which provides information about the presence of an aerosol plume in the optical beam and the typical aerosol size. As shown in FIG. 4A, the fine pesticide aerosol exhibits a stronger extinction contribution at the higher frequency (shorter wavelength) end of the spectrum, which results in the appearance of slightly negative slope in the baseline offset. In this example, the pesticide also employed a hydrocarbon carrier, which exhibits a C—H stretching mode in the 3000-cm⁻¹ spectral region.

The second aspect of FIG. 4A relates to the derivative-like features in the 900 to 1100 cm⁻¹ fingerprint region. These features are a result of the interdependence between the imaginary and real parts of the complex refractive index in the vicinity of an absorption feature of the aerosolized material. The specificity of this unique feature correlates well with the rise in the baseline offset, which facilitates the identification of the released malathion. FIG. 4B shows the spectrum shown in FIG. 4A expanded in the fingerprint region of the mid-infrared spectral region.

In this example, a small amount of gas phase carbon monoxide also was measured from a distant power generator.

### Example 2

In addition to measuring the apparent absorbance spectrum of aerosolized malathion as described immediately hereinabove in Example 1 and shown in FIG. 4A, a plume of dust particles and plumes comprising mixtures of different amounts of malathion adsorbed on dust also were released in separate experiments. An enlargement of the fingerprint spectral region for these releases is shown in FIG. 6, which provides the apparent absorbance spectrum of aerosolized malathion in the absence of dust particles (thick solid line); the apparent absorbance spectrum of malathion adsorbed on airborne dust particles (thin solid line); and the apparent absorbance spectrum of airborne dust particles (dotted line).

The apparent absorbance spectrum of malathion adsorbed on airborne dust particles (thin solid line) exhibits a derivative-like feature similar to aerosolized malathion in the 950- to 1100-cm⁻¹ region (thick solid line) and dust extinction features (dotted line). The presence of both features in this spectrum of malathion adsorbed on airborne dust particles demonstrates the capabilities of the presently disclose ORS method to remotely identify low vapor pressure compounds in the liquid aerosolized phase as well as low vapor pressure compounds adsorbed on airborne particulate matter. This behavior was observed in each of the different mixtures of malathion adsorbed on dust particles. The apparent absorbance spectrum of these mixtures exhibited a monotonic increase in the specificity of the derivative-like feature with increasing amount of malathion adsorbed on the dust particles.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for monitoring for a low vapor pressure compound in the atmosphere, the method comprising:
(a) providing an instrument adapted for emitting modulated infrared radiation along a monitoring path;
(b) providing at least one detector disposed so as to detect the modulated infrared radiation emitted by the instrument, wherein the detector is capable of producing a signal indicative of the apparent absorption spectrum of the low vapor pressure compound;
(c) positioning the instrument such that the emitted modulated infrared radiation traverses the monitoring path;
(d) measuring the apparent absorption spectrum of the low vapor pressure compound, wherein the apparent absorption spectrum exhibits two or more characteristics selected from the group consisting of:
(i) one or more absorption bands;
(ii) one or more derivative-like features;
(iii) one or more wavelength dependent baseline offsets; and
(iv) combinations thereof; and
(e) correlating the two or more characteristics to provide one of:
(i) a detection;
(ii) an identification;
(iii) a quantification; and
(iv) combinations thereof;
of one or more low vapor pressure compounds to monitor the one or more low vapor pressure compounds in the atmosphere.

2. The method of claim 1, wherein the low vapor pressure compound comprises a physical state, wherein the physical state is selected from the group consisting of a vapor phase, an aerosol phase, adsorbed on airborne particulate matter, and combinations thereof.

3. The method of claim 1, wherein the low vapor pressure compound comprises a toxic chemical.

4. The method of claim 3, wherein the toxic chemical is selected from the group consisting of an industrial toxic chemical, an agricultural chemical, a chemical warfare agent, and a bioaerosol.

5. The method of claim 3, wherein the toxic chemical comprises an organophosphate toxic chemical.

6. The method of claim 1, wherein the instrument comprises an active open-path Fourier transform infrared (OP-IR) spectrometer system.

7. The method of claim 6, wherein the open-path infrared spectrometer system comprises an open-path Fourier transform infrared spectrometer system.

8. The method of claim 7, wherein the open-path Fourier transform infrared spectrometer system comprises a monostatic configuration.

9. The method of claim 1, wherein the instrument comprises a pulsed quantum cascade (QC) laser infrared radiation source.

10. The method of claim 1, wherein the instrument has a spectral range of at about 700 cm⁻¹ to about 5000 cm⁻¹.

11. The method of claim 1, wherein the detector is selected from the group consisting of a photoconducting detector and a thermal detector.

12. The method of claim 1, wherein the monitoring path is positioned along a perimeter of a facility.

13. The method of claim 12, wherein the facility is a facility having one or more toxic chemicals disposed therein.

14. The method of claim 12, wherein the facility houses one or more human occupants.

15. The method of claim 1, wherein the one or more absorption bands indicates the presence of one or more a low vapor pressure compounds in a vapor phase in the monitoring path.

16. The method of claim 1, wherein the one or more derivative-like features indicates the presence of one or more low vapor pressure compounds in one of an aerosol phase, a particle phase, and combinations thereof in the monitoring path.

17. The method of claim 1, wherein the one or more wavelength dependent baseline offsets indicates the presence of one or more low vapor pressure compound in one of an aerosol phase, a particle phase, and combinations thereof in the monitoring path.

18. The method of claim 1, wherein the correlating of the two or more characteristics indicates the presence of one or more low vapor pressure compounds in one of a vapor phase, an aerosol phase, a particle phase, and combinations thereof in the monitoring path.

19. The method of claim 1, wherein the correlating of the two or more characteristics is performed in real-time.

20. A system for monitoring for one or more low vapor pressure compounds in the atmosphere, the system comprising:
(a) an instrument adapted for emitting modulated infrared radiation along a monitoring path;
(b) at least one detector disposed so as to detect the modulated infrared radiation emitted by the instrument, wherein the detector is capable of producing a signal indicative of the apparent absorption spectrum of the low vapor pressure compound, and wherein the apparent absorption spectrum exhibits two or more characteristics selected from the group consisting of:
(i) one or more absorption bands;
(ii) one or more derivative-like features;
(iii) one or more wavelength dependent baseline offset; and
(iv) combinations thereof;
(c) a memory in which a plurality of machine instructions are stored; and
(d) at least one processor that is coupled to the at least one detector and the memory, wherein the processor is capable of executing the plurality of machine instructions stored in the memory, causing the processor to:
(i) record the signal indicative of the apparent absorption spectrum of the low vapor pressure compound, wherein the apparent absorption spectrum exhibits two or more characteristics selected from the group consisting of one or more absorption bands, one or more derivative-like features; one or more wavelength dependent baseline offsets; and combinations thereof; and
(ii) correlate the two or more characteristics to provide one of a detection; an identification; a quantification; and combinations thereof of one or more low vapor pressure compounds to monitor one or more low vapor pressure compounds in the atmosphere.

21. The system of claim 20, wherein the instrument comprises an active open-path Fourier transform infrared (OP-FTIR) spectrometer system.

22. The system of claim 21, wherein the open-path Fourier transform infrared spectrometer system comprises a monostatic configuration.

23. The system of claim 20, wherein the instrument comprises a pulsed quantum cascade (QC) laser infrared radiation source.

24. The system of claim 20, wherein the instrument has a spectral range of at about 700 cm⁻¹ to about 5000 cm⁻¹.

25. The system of claim 20, wherein the detector is selected from the group consisting of a photoconducting detector and a thermal detector.

26. The system of claim 20, wherein the instrument is transportable.

27. A computer program product comprising computer-executable instructions embodied in a computer-readable medium for performing steps comprising:
(a) inputting a signal indicative of the apparent absorption spectrum of a low vapor pressure compound, wherein the apparent absorption spectrum exhibits two or more characteristics selected from the group consisting of one or more absorption bands, one or more derivative-like features, one or more wavelength dependent baseline offsets, and combinations thereof; and
(b) correlating the two or more characteristics to monitor for one or more low vapor pressure compounds in the atmosphere.
